(19) United States
(12) Patent Application Publication
(10) Pub. No.: US 2010/0113908 A1
(43) Pub. Date: May 6, 2010

(54) System And Method For Facilitating Observation Of Monitored Physiologic Data

(75) Inventors: Steve Vargas, Sun Valley, CA (US); Robin Boyce, Pleasanton, CA (US); Li Li, Milpitas, CA (US); Hui Wang, San Ramon, CA (US); Scott Amundson, Oakland, CA (US); James Ochs, Seattle, WA (US); Tonia Madere, Stockton, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/609,304

(22) Filed: Oct. 30, 2009

Related U.S. Application Data
(60) Provisional application No. 61/110,299, filed on Oct. 31, 2008.

(51) Int. Cl. A61B 5/145

(52) U.S. Cl. 600/364

(57) ABSTRACT

Present embodiments are directed to a system and method capable of detecting and graphically indicating physiologic patterns in patient data. For example, present embodiments may include a monitoring system that includes a monitor capable of receiving input relating to patient physiological parameters and storing historical data related to the parameters. Additionally, the monitoring system may include a screen capable of displaying the historical data corresponding to the patient physiological parameters. Further, the monitoring system may include a pattern detection feature capable of analyzing the historical data to detect a physiologic pattern in a segment of the historical data and capable of initiating a graphical indication of the segment on the screen when the physiologic pattern is present in the segment.

### RELATED APPLICATION

This application claims the benefit of U.S. Provisional Application No. 61/110,299 filed Oct. 31, 2008, which application is hereby incorporated herein by reference.

### BACKGROUND OF THE INVENTION

1. Technical Field

The present disclosure relates generally to user-interface applications for patient monitoring devices. In particular, present embodiments relate to display features that facilitate observation of monitored physiological data with patient monitoring instruments.

2. Description of the Related Art

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Patient monitors include medical devices that facilitate measurement and observation of patient physiological data. For example, pulse oximeters are a type of patient monitor. A typical patient monitor cooperates with a sensor to detect and display a patient's vital signs (e.g., temperature, pulse rate, or respiratory rate) and/or other physiological measurements (e.g., water content of tissue, or blood oxygen level) for observation by a user (e.g., clinician). For example, pulse oximeters are generally utilized with related sensors to detect and monitor a patient's functional oxygen saturation of arterial hemoglobin (i.e., SpO₂) and pulse rate. Other types of patient monitors may be utilized to detect and monitor other physiological parameters. The use of patient monitors may improve patient care by facilitating supervision of a patient without continuous attendance by a human observer (e.g., a nurse or physician).

A patient monitor may include a screen that displays information relating to operation and use of the patient monitor. A typical patient monitor screen may display operational data that is instructive and that facilitates operation of the monitor by a user. For example, the operational data may include status indicators and instructional data relating to the monitor itself and/or monitor applications (e.g., a power indicator, an alarm silenced icon, and a battery low indicator). The screen may also display measurement data from a patient being monitored. For example, the measurement data may include information relating to a physiological feature of the patient being monitored. Specifically, the screen may display a graph or trend (e.g., a pulse rate trend and/or a plethysmographic waveform) of data relating to particular measured physiological parameters. Such trends include historical data that may span short or long periods of time in which particular parameters (e.g., SpO₂ and/or pulse rate) being trended were observed. This historical data can be beneficial for handling and detecting patient issues. However, analysis of this historical information can be inconvenient due to the quantity of the information. Further, such analysis can be difficult because certain aspects of the information are difficult for a user to detect.

### BRIEF DESCRIPTION OF THE DRAWINGS

Advantages of present embodiments may become apparent upon reading the following detailed description and upon reference to the drawings in which:

FIG. 1 is a perspective view of a patient monitor in accordance with an exemplary embodiment of the present disclosure;

FIG. 2 is a perspective view of the patient monitor in a system with separate devices in accordance with an exemplary embodiment of the present disclosure;

FIG. 3 is a representation of a display including a trend of physiological data with labeled components in accordance with an exemplary embodiment of the present disclosure;

FIG. 4 is a representation of a display including a trend of physiological data that exhibits a detected pattern in accordance with an exemplary embodiment of the present disclosure;

FIG. 5 is a block diagram of an electronic device in accordance with an exemplary embodiment of the present disclosure;

FIG. 6 is a graph of SpO₂ trend data with an upper band and lower band based on mean and standard deviation values in accordance with an exemplary embodiment of the present disclosure;

FIG. 7 is an exemplary graph including an SpO₂ trend that contains a ventilatory instability SpO₂ pattern and a trend of the resulting saturation pattern detection index in accordance with an exemplary embodiment of the present disclosure;

FIG. 8 is a representation of a display wherein portions of a trend are distinguished by different graphic features to designate a position in time in accordance with an exemplary embodiment of the present disclosure;

FIG. 9 is a representation of a display wherein detected patterns in a trend are highlighted in accordance with an exemplary embodiment of the present disclosure;

FIG. 10 is a display screen including various textual and graphical indicators to facilitate user review of areas of interest in historical trend data in accordance with an exemplary embodiment of the present disclosure;

FIG. 11 is a front view of a control panel in accordance with an exemplary embodiment of the present disclosure;

FIG. 12 is a front view of a control panel in accordance with an exemplary embodiment of the present disclosure; and

FIG. 13 is a front view of a control panel in accordance with an exemplary embodiment of the present disclosure.

### DETAILED DESCRIPTION OF SPECIFIC EMBODIMENTS

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Embodiments of the present disclosure are directed to a user-interface feature for a patient monitoring device. Specifically, present embodiments include a display control feature that facilitates observation and analysis of historical trend data. The display control feature automatically finds and displays particular designated events in the historical data so that the events may be analyzed by a user. These events may include alarms, detected patterns (e.g., ventilatory instability or desaturation patterns), maximum values, minimum values, markers inserted automatically or by users, and so forth. For example, the display control feature may enable a user to automatically scroll, jump, or snap to a particular event by pressing a scroll button, turning a knob, or selecting an icon on a navigable menu. Thus, a user may utilize present embodiments to avoid the inefficiency of methodically scrolling through large amounts (e.g., hours) of trend data (e.g., a continuous chart of SpO₂ values) in search of patterns (e.g., a desaturation pattern) or other events (e.g., alarms). Indeed, in accordance with present embodiments, the user may simply utilize an activation mechanism (e.g., a control knob, button, or selectable menu) that coordinates with the display control feature to display events. For example, a control knob may be turned or a button may be pressed to display the last detected desaturation pattern in a trend of SpO₂ data. Further, additional turns of the knob or presses of the button may allow the user to cycle through all or a portion of the detected desaturation patterns and/or other events.

Additionally, present embodiments may facilitate observation of certain events (e.g., SpO₂ patterns) displayed on a monitor's user-interface by graphically drawing attention to areas of interest in trend data and by providing graphic indicators that relate to the status of certain features. For example, specific portions of a graphical representation of physiologic data may be highlighted or flashed to draw attention to a particular series of data points because the data points have been identified as corresponding to a particular pattern. As a specific example, a monitor in accordance with present embodiments may display a graphical trend of data values received from a sensor, wherein the data values correspond to physiologic data measurements from a patient. If a series of the data values is identified as corresponding to ventilatory instability, present embodiments may flash or highlight the portion of the graphical trend that has been identified as having a pattern associated with the ventilatory instability. Present embodiments may also facilitate identification of the time of occurrence of events in the monitoring history by placing a time scale along the trend graph of the data. For example, the time scale may include onset and offset times for the section of data that is being viewed and/or the portion of data that has been identified as corresponding to a particular physiologic pattern.

Further, present embodiments may include one or more graphic features that are actively representative of a status of pattern detection or a level (e.g., a percentage of an alarm level) of a detected occurrence. Such graphic features may provide an active representation of a gradual build up of indicators that correspond to identification of a particular pattern or that are indicative of a severity level of an identified condition. Indeed, present embodiments may utilize an accumulation of data indicators to identify a physiologic pattern or a severity level of a particular event, and the graphic feature may gradually change as observed indications accumulate. For example, in accordance with present embodiments, ventilatory instability may be detected when a fixed number of certain data features have been detected within a time period. Thus, a percentage value associated with ventilatory instability detection may be identified by dividing the number of detected data features by the fixed number utilized for identification of a ventilatory instability pattern, and the percentage may be represented in a dynamic graphic (e.g., a status bar). As a specific example, a graphic displayed as a triangle outline may gradually fill in the triangle outline from the bottom with coloring as certain indicators of a particular pattern accumulate. Thus, the triangle graphic may be completely filled in with color when the pattern is actually confirmed. Likewise, the triangle may empty of color when certain aspects are reduced. Similarly, a graphic may gradually fill or empty as certain severity thresholds or indexes of a particular event are reached.

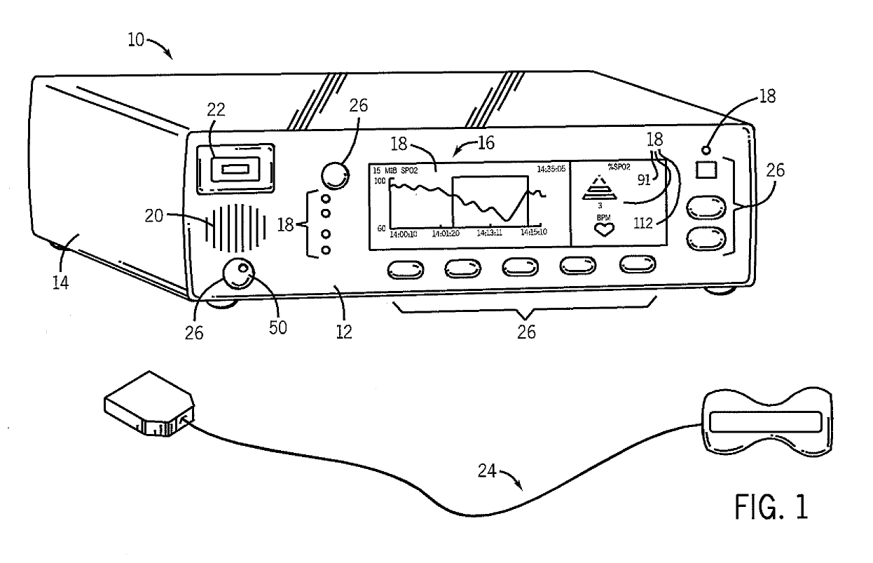

FIG. 1 is a perspective view of a patient monitor 10 in accordance with an exemplary embodiment of the present disclosure. Specifically, the patient monitor 10 illustrated by FIG. 1 is a pulse oximeter that is configured to detect and monitor blood oxygen saturation levels, pulse rate, and so forth. It should be noted that while the illustrated embodiment includes a pulse oximeter, other embodiments may include different types of patient monitors 10. For example, the patient monitor 10 may be representative of a vital signs monitor, a critical care monitor, an obstetrical care monitor, or the like.

The illustrated patient monitor 10 includes a front panel 12 coupled to a body 14 of the monitor 10. The front panel 12 includes a display screen 16 and various indicators 18 (e.g., indicator lights and display screen graphics) that facilitate operation of the monitor 10 and observation of a patient's physiological metrics (e.g., pulse rate). Some of the indicators 18 are specifically provided to facilitate monitoring of a patient's physiological parameters. For example, the indicators 18 may include representations of the most recently measured values for SpO₂, pulse rate, index values, and pulse amplitude. Other indicators 18 may be specifically provided to facilitate operation of the monitor 10. For example, the indicators 18 may include an A/C power indicator, a low battery indicator, an alarm silence indicator, a mode indicator, and so forth. The front panel 12 may also include a speaker 20 for emitting audible indications (e.g., alarms), a sensor port 22 for coupling with a sensor 24 (e.g., a temperature sensor, a pulse oximeter sensor) and other monitor features.

Additionally, the front panel 12 may include various activation mechanisms 26 (e.g., buttons and switches) to facilitate management and operation of the monitor 10. For example, the front panel 12 may include function keys (e.g., keys with varying functions), a power switch, adjustment buttons, an alarm silence button, and so forth. It should be noted that in other embodiments, the indicators 18 and activation mechanisms 26 may be arranged on different parts of the monitor 10. In other words, the indicators 18 and activation mechanisms 26 need not be located on the front panel 12. Indeed, in some embodiments, activation mechanisms 26 are virtual representations in a display or actual components disposed on separate devices.

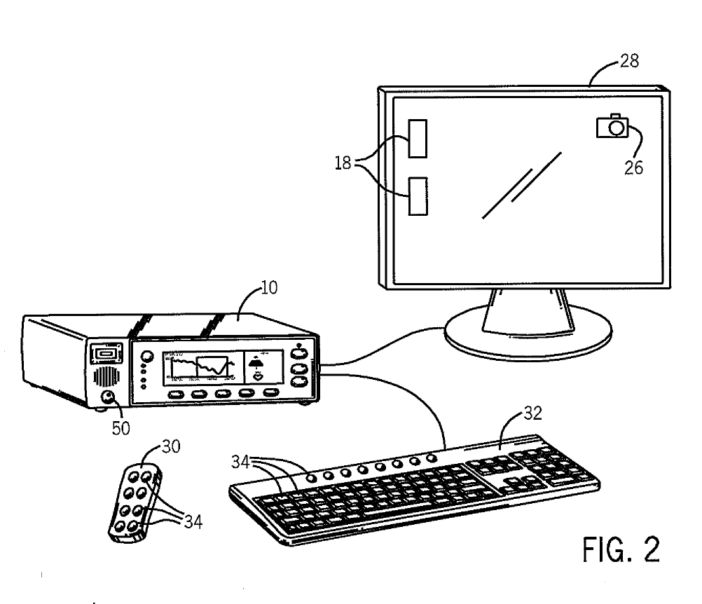

In some embodiments, as illustrated in FIG. 2, the monitor 10 may cooperate with separate devices, such as a separate screen 28, a wireless remote 30, and/or a keyboard 32. These separate devices may include some of the indicators 18 and activation mechanisms 26 described above. For example, buttons 34 on the remote 30 and/or keyboard 32 may operate as activation mechanisms 26. Specifically, for example, the buttons 34 may cause the monitor 10 to perform specific operations (e.g., power up, adjust a setting, silence an alarm) when actuated on the separate device. Similarly, the indicators 18 and/or activation mechanisms 26 may not be directly disposed on the monitor 10. For example, the indicators 18 may include icons, indicator lights, or graphics on the separate screen 28 (e.g., a computer screen). Further, the activation mechanisms 26 may include programs or graphic features that can be selected and operated via a display. It should be noted that the separate screen 28 and/or the keyboard 32 may communicate directly or wirelessly with the monitor 10.

As briefly set forth above, embodiments of the present disclosure include a display control feature that facilitates observation and analysis of historical data. This display control feature may include software or hardware, as well as an activation mechanism to operate the display control feature. For example, FIGS. 1 and 2 include a knob 50 that may be utilized to operate the display control feature. The display control feature may facilitate a user's observation of certain events (e.g., metrics and indications) by eliminating or reducing the time and effort required for a user to find the events by scanning through the data (e.g., trend data). For example, the display control feature may enable a user to turn the knob 50 or to use some other activation mechanism to cause the view provided by the monitor 10 to automatically snap or jump to certain events. In other words, present embodiments may allow a user to snap or jump directly to screens displaying certain events (e.g., alarms, detected patterns, maximum values, minimum values) by activating the display control feature. Indeed, a user may select a particular type of event or particular types of events to jump to and/or skip over. In one embodiment, a user can turn the knob 50 to scroll through various options and then push the knob 50 to select a particular option (e.g., jump to the latest detected desaturation pattern) that causes the display to jump to certain events. In some embodiments, the knob 50 may be replaced by other activation mechanisms. For example, a user may activate the display control feature by pressing a button and/or maneuvering a roller ball. It should be noted that the data to which the monitor 10 snaps or jumps may be displayed by the monitor 10 on the display screen 16 and/or the separate screen 28. Features related to identifying events and then jumping or snapping to the identified events will be discussed in further detail below.

In one embodiment, the monitor 10 may detect and label certain events that can later be readily accessed using the display control feature. Indeed, the events may be continuously detected and labeled by a detection feature of the monitor 10. Additionally, a user may designate certain data points, time periods, and so forth as events. For example, a user may select certain data points for review by highlighting and manually labeling the data. Once such events have been identified, a user may jump or cycle to displays that illustrate the detected events by activating (e.g., depressing, or rotating) the activation mechanism (e.g., knob 50) of the display control feature.

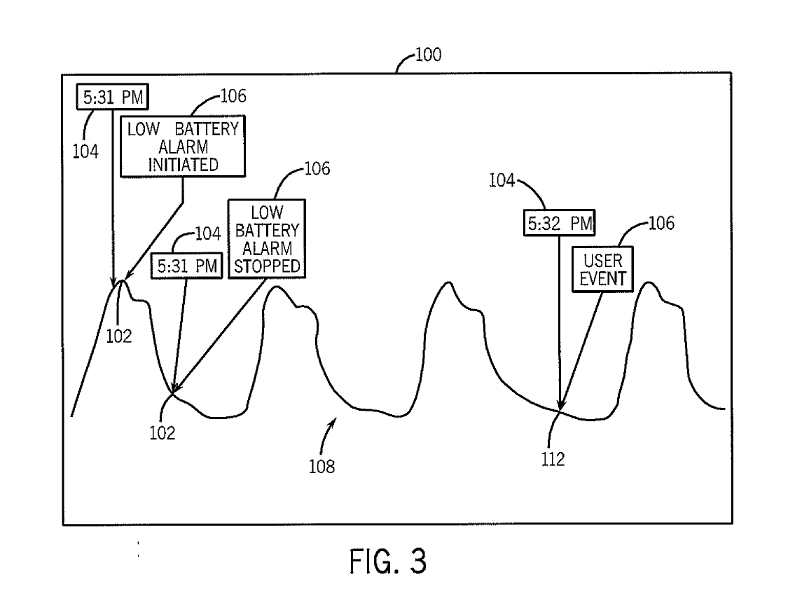

In a specific example, as illustrated in the exemplary display 100 in FIG. 3, the monitor 10 may automatically label the moment at which an alarm 102 was initiated by designating the alarm 102 with a timestamp 104 and/or graphic indicator 106, for example, at the corresponding location of the alarm 102 on a trend 108. Deactivation of the alarm 102 may also be designated on the trend 108. It should be noted that the alarm 102 may correspond to detected physiological data (e.g., high temperature or low saturation) or any other type of alarm condition (e.g., low battery or sensor off). A user may also manually designate an event, as illustrated by user designated event 112. As with automatically detected events (e.g., alarm 102), such user designated events may also be automatically timestamped.

In some embodiments, the monitor 10 may detect patterns in data (e.g., physiological data) that correspond to certain conditions. For example, present embodiments may detect a cluster of desaturation data or a desaturation pattern that is indicative of ventilatory instability in the patient being monitored. In some embodiments, ventilatory instability may be defined as a significant cyclical reduction in airflow, as measured by a nasal airflow sensor, accompanied by a reduction in chest and/or abdomen wall movement. Such reductions in airflow may cause a patient's SpO₂ to cyclically rise and fall as the patient begins to desaturate due to lack of oxygen and then subsequently recover (i.e., re-saturate). Thus, such SpO₂ cycles may be indicative of ventilatory instability. One example of ventilatory instability is sleep apnea.

Upon detecting such patterns, the monitor 10 may label (e.g., timestamp, textually indicate, highlight, or flash) the graphical representation of the initial portion of the pattern and the end portion of the pattern. In other words, the monitor may 10 provide an indication of the pattern data from where the pattern begins to where it ends once the pattern has been determined to exist. For example, in one embodiment, a pattern portion of a trend may be displayed in reverse video (e.g., flashing or highlighted) or indicated with a particular color (e.g., highlighted or colored with red to indicate high relevance, yellow to indicate medium relevance, and green to indicate low relevance). In another embodiment, the pattern portion of the trend may be displayed with a line having a distinguishing thickness or color. Further, the monitor 10 may essentially diagnose the pattern by labeling it with specific text or other graphical features based on a database of correlations between labels and detected patterns.

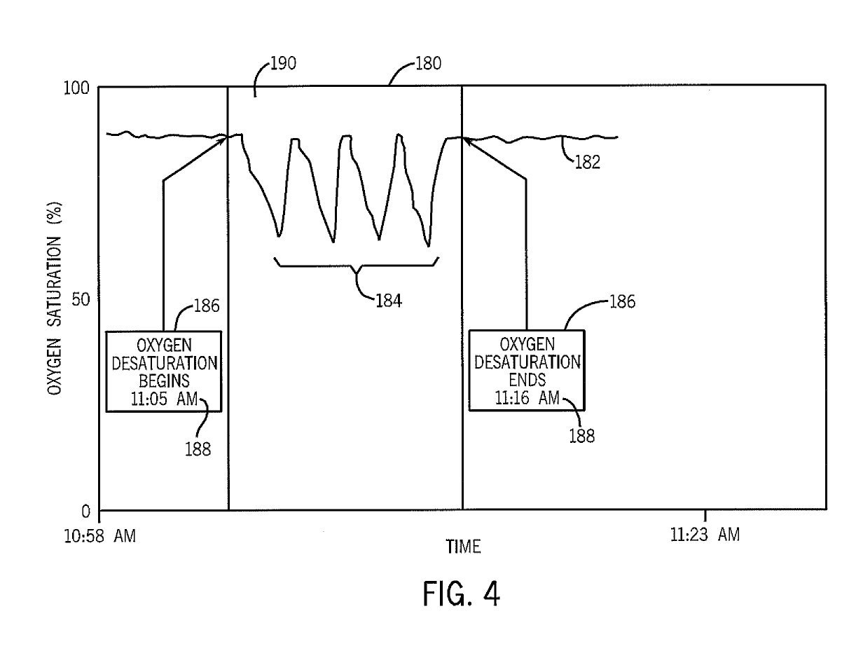

FIG. 4 is a representation of a display 180 that includes a trend 182 of oxygen saturation over time. As illustrated in FIG. 4, the monitor 10 may detect a cluster or pattern 184 of desaturation data, which the monitor 10 may determine is likely indicative of sleep apnea or some other issue. The monitor 10 may then label the pattern 184 with a textual graphic 186 and a timestamp 188 indicating a beginning and end of the detected pattern 184. Further, the monitor 10 may highlight or flash the pattern, as indicated by block 190, or utilize some other graphical indicator. Such labeling and/or indication may facilitate rapid diagnosis of a patient by a clinician. For example, the clinician may use present embodiments to simply snap or jump to a display including the pattern 184 (e.g., indication of sleep apnea or ventilation instability) by activating the display control feature (e.g., pressing a button), and the graphic indicators may draw the users attention to facilitate diagnosis.

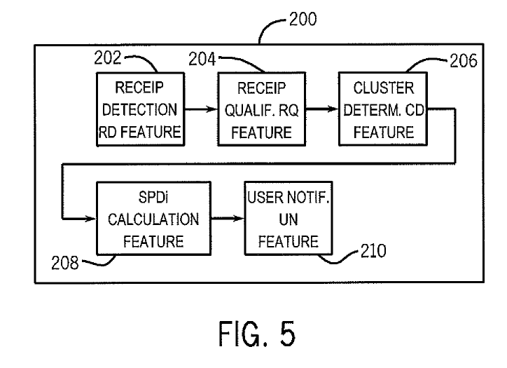

In order to graphically or textually indicate the patterns in SpO₂ trend data (e.g., saturation patterns indicative of ventilatory instability), as discussed above, the patterns must first be detected. Accordingly, present embodiments may include code stored on a tangible, computer-readable medium (e.g., a memory) and/or hardware capable of detecting the presence of a saturation pattern in a series of physiologic data. For example, FIG. 5 is a block diagram of an electronic device or pattern detection feature in accordance with present embodiments. The electronic device is generally indicated by the reference number 200. The electronic device 200 (e.g., an SpO₂ monitor and/or memory device) may comprise various subsystems represented as functional blocks in FIG. 5. Those of ordinary skill in the art will appreciate that the various functional blocks shown in FIG. 5 may comprise hardware elements (e.g., circuitry), software elements (e.g., computer code stored on a hard drive) or a combination of both hardware and software elements. For example, each functional block may represent software code and/or hardware components that are configured to perform portions of an algorithm in accordance with present embodiments. Specifically, in the illustrated embodiment, the electronic device 200 includes a reciprocation detection (RD) feature 202, a reciprocation qualification (RQ) feature 204, a cluster determination (CD) feature 206, a saturation pattern detection index (SPDi) calculation feature 208, and a user notification (UN) feature 210. Each of these components and the coordination of their functions will be discussed in further detail below.

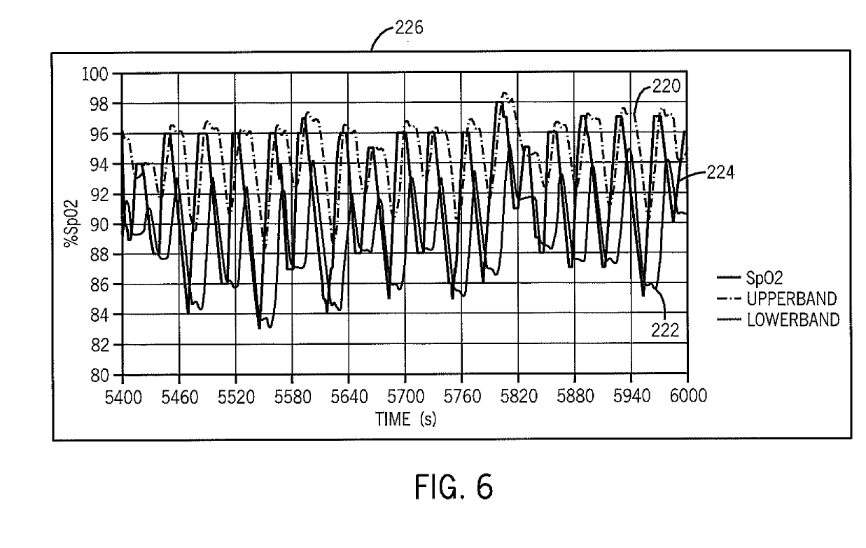

The RD feature 202 may be capable of performing an algorithm for detecting reciprocations in a data trend. Specifically, the algorithm of the RD feature 202 may perform a statistical method to find potential reciprocation peaks and nadirs in a trend of SpO₂ data. A nadir may be defined as a minimum SpO₂ value in a reciprocation. The peaks may include a rise peak (e.g., a maximum SpO₂ value in a reciprocation that occurs after the nadir) and/or a fall peak (e.g., a maximum SpO₂ value in a reciprocation that occurs before the nadir). Once per second, the RD feature 202 may calculate a 12 second rolling mean and standard deviation of the SpO₂ trend. Further, based on these mean and standard deviation values, an upper band 220 and lower band 222 with respect to an SpO₂ trend 224, as illustrated by the graph 226 in FIG. 6, may be calculated as follows:

Upper Band=mean+standard deviation;

Lower Band=mean−standard deviation.

Once the upper band 220 and lower band 222 have been determined, potential reciprocation peaks and nadirs may be extracted from the SpO₂ trend 224 using the upper band 220 and the lower band 224. Indeed, a potential peak may be identified as the highest SpO₂ point in a trend segment which is entirely above the upper band 220. Similarly, a potential nadir may be identified as the lowest SpO₂ point in a trend segment that is entirely below the lower band 222. In other words, peaks identified by the RD feature 202 may be at least one standard deviation above the rolling mean, and nadirs identified by the RD feature 202 may be at least one standard deviation below the mean. If there is more than one minimum value below the lower band 222, the last (or most recent) trend point may be identified as a nadir. If more than one maximum value is above the upper band 220, the point identified as a peak may depend on where it is in relation to the nadir. For example, regarding potential peaks that occur prior to a nadir (e.g., fall peaks), the most recent maximum trend point may be used. In contrast, for peaks that occur subsequent to a nadir (e.g., rise peaks), the first maximum point may be used. In the example trend data represented in FIG. 6, a peak and nadir is detected approximately every 30-60 seconds.

In one embodiment, a window size for calculating the mean and standard deviation may be set based on historical values (e.g., average duration of a set number of previous reciprocations). For example, in one embodiment, a window size for calculating the mean and standard deviation may be set to the average duration of all qualified reciprocations in the last 6 minutes divided by 2. In another embodiment, a dynamic window method may be utilized wherein the window size may be initially set to 12 seconds and then increased as the length of qualified reciprocations increases. This may be done in anticipation of larger reciprocations because reciprocations that occur next to each other tend to be of similar shape and size. If the window remained at 12 seconds, it could potentially be too short for larger reciprocations and may prematurely detect peaks and nadirs. The following equation or calculation is representative of a window size determination, wherein the output of the filter is inclusively limited to 12-36 seconds, and the equation is executed each time a new reciprocation is qualified:

```
If no qualified reciprocations in the last 6 minutes:
        Window Size = 12 (initial value)
else:
        RecipDur = ½ * current qualified recip duration +
        ½ * previousRecipDur
        Window Size = bound(RecipDur,12,36).
```

With regard to SpO₂ signals that are essentially flat, the dynamic window method may fail to find the three points (i.e., a fall peak, a rise peak, and a nadir) utilized to identify a potential reciprocation. Therefore, the RD feature 202 may limit the amount of time that the dynamic window method can search for a potential reciprocation. For example, if no reciprocations are found in 240 seconds plus the current dynamic window size, the algorithm of the RD feature 202 may timeout and begin to look for potential reciprocations at the current SpO₂ trend point and later. The net effect of this may be that the RD feature 202 detects potential reciprocations less than 240 seconds long.

Once potential peaks and nadirs are found using the RD feature 202, the RQ feature 204 may pass the potential reciprocations through one or more qualification stages to determine if a related event is caused by ventilatory instability. A first qualification stage may include checking reciprocation metrics against a set of limits (e.g., predetermined hard limits). A second qualification stage may include a linear qualification function. In accordance with present embodiments, a reciprocation may be required to pass through both stages in order to be qualified.

As an example, in a first qualification stage, which may include a limit-based qualification, four metrics may be calculated for each potential reciprocation and compared to a set of limits. Any reciprocation with a metric that falls outside of these limits may be disqualified. The limits may be based on empirical data. For example, in some embodiments, the limits may be selected by calculating the metrics for potential reciprocations from sleep lab data where ventilatory instability is known to be present, and then comparing the results to metrics from motion and breathe-down studies. The limits may then be refined to filter out true positives.

The metrics referred to above may include fall slope, magnitude, slope ratio, and path length ratio. With regard to fall slope, it may be desirable to limit the maximum fall slope to filter out high frequency artifact in the SpO₂ trend, and limit the minimum fall slope to ensure that slow SpO₂ changes are not qualified as reciprocations. Regarding magnitude, limits may be placed on the minimum magnitude because of difficulties associated with deciphering the difference between ventilatory instability reciprocations and artifact reciprocations as the reciprocation size decreases, and on the maximum magnitude to avoid false positives associated with sever artifact (e.g., brief changes of more than 35% SpO₂ that are unrelated to actual ventilatory instability). The slope ratio may be limited to indirectly limit the rise slope for the same reasons as the fall slope is limited and because ventilatory instability patterns essentially always have a desaturation rate that is slower than the resaturation (or recovery) rate. The path length ratio may be defined as Path Length/((Fall Peak−Nadir)+(Rise Peak−Nadir)), where Path Length=Σ|Current SpO₂ Value−Previous SpO₂ value| for all SpO₂ values in a reciprocation, and the maximum path length ratio may be limited to limit the maximum standard deviation of the reciprocation, which limits high frequency artifact. The following table (Table I) lists the above-identified metrics along with their associated equations and the limits used in accordance with one embodiment:

TABLE I

| Metric | Equation | Minimum | Maximum |
|---|---|---|---|
| Fall Slope | (Nadir − Fall Peak)/Time | −1.6 | −0.08 |
| | between Fall Peak and Nadir | (Fast | (Fast |
| | | Response | Response |
| | | Mode) | Mode) |
| | | −1 | −0.05 |
| | | (Normal | (Normal |
| | | Response | Response Mode) |
| | | Mode) | |
| Magnitude | Max(Rise Peak, Fall Peak) − | 3 | 35 |
| | Nadir | | |
| Slope | \|Fall Slope/Rise Slope\| | 0.05 | 1.75 |
| Ratio | | | |
| Path | Path Length = _{Σ}\|Current | N/A | 2 |
| Length | SpO2 Value − Previous SpO2 | | |
| Ratio | Value\| for all SpO2 values in | | |
| | a Reciprocation. | | |
| | Path Length Ratio = Path | | |
| | Length/((Fall Peak − Nadir) + | | |
| | (Rise Peak − Nadir)) | | |

As indicated in Table I above, an oximetry algorithm in accordance with present embodiments may operate in two response modes: Normal Response Mode or Fast Response Mode. The selected setting may change the SpO₂ filtering performed by the oximetry algorithm, which in turn can cause changes in SpO₂ patterns. Therefore a saturation pattern detection feature may also accept a response mode so that it can account for the different SpO₂ filtering. Table I indicates values associated with both types of response mode with regard to the Fall Slope values.

A second qualification stage of the RQ feature 204 may utilize a object reciprocation qualification feature. Specifically, the second qualification stage may utilize a linear qualification function based on ease of implementation, efficiency, and ease of optimization. The equation may be determined by performing a least squares analysis. For example, such an analysis may be performed with MATLAB®. The inputs to the equation may include the set of metrics described below. The output may be optimized to a maximum value for patterns where ventilatory instability is known to be present. The equation may be optimized to output smaller values (e.g., 0) for other data sets where potential false positive reciprocations are abundant.

To simplify optimization, the equation may be factored into manageable sub-equations. For example, the equation may be factored into sub-equation 1, sub-equation D, and sub-equation 2, as will be discussed below. The output of each sub-equation may then be substituted into the qualification function to generate an output. The outputs from each of the sub-equations may not be utilized to determine whether a reciprocation is qualified in accordance with present embodiments. Rather, an output from a full qualification function may be utilized to qualify a reciprocation. It should be noted that the equations set forth in the following paragraphs describe one set of constants. However, separate sets of constants may be used based on the selected response mode. For example, a first set of constants may be used for the Normal Response Mode and a second set of constants may be used for the Fast Response Mode.

Preprocessing may be utilized in accordance with present embodiments to prevent overflow for each part of the qualification function. The tables (Tables II-VII) discussed below, which relate to specific components of the qualification function may demonstrate this overflow prevention. Each row in a table contains the maximum value of term which is equal to the maximum value of the input variable multiplied by the constant, wherein the term “maximum” may refer to the largest possible absolute value of a given input. Each row in a table contains the maximum intermediate sum of the current term and all previous terms. For example, a second row may contain the maximum output for the second term calculated, as well as the maximum sum of terms 1 and 2. It should be noted that the order of the row may match the order that the terms are calculated by the RQ feature 204. Further, it should be noted that in the tables for each sub-equation below, equations may be calculated using temporary signed 32-bit integers, and, thus, for each row in a table where the current term or intermediate term sum exceeds 2147483647 or is less than −2147483647 then an overflow/underflow condition may occur.

A first sub-equation, sub-equation 1, may use metrics from a single reciprocation. For example, sub-equation 1 may be represented as follows:

Eq1Score=SlopeRatio*SrCf+PeakDiff*PdCf+FallSlope*FsCf+PathRatio*PrCf+Eq1Offset,

where SrCf, PdCf, FsCf, PrCf, and Eq1 Offset may be selected using least squares analysis (e.g., using MATLAB®). PeakDiff may be defined as equal to |Recip Fall Peak−Recip Rise Peak|. It should be noted that PeakDiff is typically not considered in isolation but in combination with other metrics to facilitate separation. For example, a true positive reciprocation which meets other criteria but has a high peak difference could be an incomplete recovery. That is, a patient's SpO₂ may drop from a baseline to a certain nadir value, but then fail to subsequently recover to the baseline. However, when used in combination with other metrics in the equation, PeakDiff may facilitate separation of two classifications, as large peak differences are more abundant in false positive data sets.

With regard to sub-equation 1, the tables (Tables II and III) set forth below demonstrate that the inputs may be preprocessed to prevent overflow. Further, the tables set forth below include exemplary limits that may be utilized in sub-equation 1 in accordance with present embodiments. It should be noted that Table II includes Fast Response Mode constants and Table III includes Normal Response Mode constants.

TABLE II

| | | | | | | Maximum | |
|---|---|---|---|---|---|---|---|
| | | Maximum | | | | Intermediate Sum | |
| | Variable | Variable | | Constant Value | Maximum Term | (sum of all previous | |
| Term | Type | Value (a) | Variable Preprocessing | (b) (Fast Mode) | Value (a * b) | rows) | Overflow |
| PeakDiff * PdCf | U8 | 100 | None. This value may | −29282 | −2928200 | −2928200 | NO |
| | | | not exceed 100 since | | | | |
| | | | the maximum SpO₂ | | | | |
| | | | value accepted is 100 | | | | |
| SlopeRatio * SrCf | U8 | 255 | None | −1534 | −391170 | −3319370 | NO |
| FallSlope * FsCf | S16 | −32768 | None | −19 | 622592 | −2696778 | NO |
| PathRatio * PrCf | U16 | 65535 | None | −7982 | −523100370 | −525797148 | NO |
| Eq1Offset | N/A | N/A | N/A | 809250 | 809250 | −524987898 | NO |

TABLE III

| | | | | | | Maximum | |
|---|---|---|---|---|---|---|---|
| | | Maximum | | Constant | | Intermediate | |
| | Variable | Variable | | Value (b) | Maximum Term | Sum (sum of all | |
| Term | Type | Value (a) | Variable Preprocessing | (Normal Mode) | Value (a * b) | previous rows) | Overflow |
| PeakDiff * PdCf | U8 | 100 | None. This value may not | −33311 | −3331100 | −3331100 | NO |
| | | | exceed 100 since the | | | | |
| | | | maximum SpO2 value | | | | |
| | | | accepted is 100 | | | | |
| SlopeRatio * SrCf | U8 | 255 | None | −2151 | −548505 | −3879605 | NO |
| FallSlope * FsCf | S16 | −32768 | None | −706 | 23134208 | 19254603 | NO |
| PathRatio * PrCf | U16 | 65535 | None | −6178 | −404875230 | −385620627 | NO |
| Eq1Offset | N/A | N/A | N/A | 576330 | 576330 | −385044297 | NO |

A second sub-equation, sub-equation D, may correspond to a difference between two consecutive reciprocations which have passed the hard limit qualifications checks, wherein consecutive reciprocations include two reciprocations that are separated by less than a defined time span. For example, consecutive reciprocations may be defined as two reciprocations that are less than 120 seconds apart. The concept behind sub-equation D may be that ventilatory instability tends to be a relatively consistent event, with little change from one reciprocation to the next. Artifact generally has a different signature and tends to be more random with greater variation among reciprocations. For example, the following equation may represent sub-equation D:

EqD=SlopeRatioDiff*SrDCf+DurationDiff*DDCf+NadirDiff*NdCf+PathLengthRatioDiff*PrDCf_{—}EqDOffset,

where, SrDCf, DDCf, NdCf, PrDCf, and EqDOffset may be selected using least squares analysis (e.g., using MATLAB®). With regard to other variables in sub-equation D, SlopeRatioDiff may be defined as |Current Recip Slope Ratio−Slope Ratio of last qualified Recipi|; DurationDiff may be defined as |Current Recip Duration−Duration of last qualified Recip|; NadirDiff may be defined as |Current Recip Nadir−Nadir value of last qualified Recip|; and PathLengthRatioDiff may be defined as |Current Recip Path Length Ratio−Path Length Ratio of last qualified Recip|.

With regard to sub-equation D, the tables (Tables IV and V) set forth below demonstrate that the inputs may be preprocessed to prevent overflow. Further, the tables set forth below include exemplary limits that may be utilized in sub-equation D in accordance with present embodiments. It should be noted that Table IV includes Fast Response Mode constants and Table V includes Normal Response Mode constants.

TABLE IV

| | | | | Constant | | Maximum | |
|---|---|---|---|---|---|---|---|
| | | Maximum | | Value | | Intermediate Sum | |
| | Variable | Variable | Variable | (b) | Maximum Term | (sum of all previous | |
| Term | Type | Value (a) | Preprocessing | (Fast Mode) | Value (a * b) | rows) | Overflow |
| EqDOffset | N/A | N/A | N/A | 885030 | 885030 | 885030 | NO |
| SlopeRatioDiff * | U8 | 255 | None | −2809 | −716295 | 168735 | NO |
| SrDCf | | | | | | | |
| DurationDiff * DDCf | U16 | 240 | The Recip detection | −2960 | −710400 | −541665 | NO |
| | | | module may only | | | | |
| | | | detect recips less than | | | | |
| | | | or equal to 240 | | | | |
| | | | seconds long | | | | |
| NadirDiff * NdCf | U8 | 100 | This value may not | −13237 | −1323700 | −1865365 | NO |
| | | | exceed 100 since the | | | | |
| | | | maximum SpO2 value | | | | |
| | | | accepted is 100 | | | | |
| PathLengthRatioDiff * | U16 | 65535 | None | −7809 | −511762815 | −513628180 | NO |
| PrDCf | | | | | | | |

TABLE V

| | | | | | | Maximum | |
|---|---|---|---|---|---|---|---|
| | | Maximum | | Constant | Maximum | Intermediate | |
| | Variable | Variable | | Value (b) | Term Value | Sum (sum of all | |
| Term | Type | Value (a) | Variable Preprocessing | (Normal Mode) | (a * b) | previous rows) | Overflow |
| EqDOffset | N/A | N/A | N/A | 847650 | 847650 | 847650 | NO |
| SlopeRatioDiff * | U8 | 255 | None | −2629 | −670395 | 177255 | NO |
| SrDCf | | | | | | | |
| DurationDiff * DDCf | U16 | 240 | The Recip detection | −4282 | −1027680 | −850425 | NO |
| | | | module may only detect | | | | |
| | | | recips less than or equal | | | | |
| | | | to 240 seconds long | | | | |
| NadirDiff * NdCf | U8 | 100 | This value may not | −11705 | −1170500 | −2020925 | NO |
| | | | exceed 100 since the | | | | |
| | | | maximum SpO2 value | | | | |
| | | | accepted is 100 | | | | |
| PathLengthRatioDiff * | U16 | 65535 | None | −7844 | −514056540 | −516077465 | NO |
| PrDCf | | | | | | | |

A third sub-equation, sub-equation 2, may combine the output of sub-equation D with the output of sub-equation 1 for a reciprocation (e.g., a current reciprocation) and a previous reciprocation. For example, the following equation may represent sub-equation 2:

Eq2Score=EqDScore*DCf+Eq1ScoreCurrent*CurrEq1Cf+Eq1ScorePrev*PrevEq1Cf,

where DCf, N1Cf, PrevEq1Cf, and Eq2Offset may be selected using least squares analysis (e.g., using MATLAB®). With regard to other variables in sub-equation 2, EqDScore may be described as the output of sub-equation D; Eq1ScoreCurrent may be described as the output of sub-equation 1 for a current reciprocation; and Eq1ScorePrev may be described as the output of sub-equation 1 for the reciprocation previous to the current reciprocation.

With regard to sub-equation 2, the tables (Tables VI and VII) set forth below demonstrate that the inputs may be preprocessed to prevent overflow. Further, the tables set forth below include exemplary limits that may be utilized in sub-equation 2 in accordance with present embodiments. It should be noted that Table VI includes Fast Response Mode constants and Table VII includes Normal Response Mode constants.

TABLE VI

| | | | | | | Maximum | |
|---|---|---|---|---|---|---|---|
| | | Maximum | | | | Intermediate Sum | |
| | Variable | Variable | | Constant Value | Maximum Term | (sum of all | |
| Term | Type | Value (a) | Variable Preprocessing | (b) (Fast Mode) | Value (a * b) | previous rows) | Overflow |
| Eq2Offset | N/A | N/A | N/A | −203800 | −203800 | −203800 | NO |
| EqDScore * DCf | S32 | −501590 | The largest output for sub- | 529 | −265341110 | −265544910 | NO |
| | | | equation D may be −513628180 | | | | |
| | | | (see Table IV). | | | | |
| | | | The input value may be | | | | |
| | | | scaled by dividing the value | | | | |
| | | | by 1024. Therefore the | | | | |
| | | | largest input value may be −501590 | | | | |
| Eq1ScorePrev * | S32 | −512683 | The largest output for sub- | 333 | −170723439 | −436268349 | NO |
| PrevEq1Cf | | | equation 1 may be −524987898 | | | | |
| | | | (see Table II). | | | | |
| | | | The input value may be | | | | |
| | | | scaled by dividing the value | | | | |
| | | | by 1024. Therefore the | | | | |
| | | | largest input value may be −512683 | | | | |
| Eq1ScoreCurrent * | S32 | −512683 | Same as previous row | 617 | −316325411 | −752593760 | NO |
| CurrEq1Cf | | | | | | | |

TABLE VII

| | | | | | | Maximum | |
|---|---|---|---|---|---|---|---|
| | | | | Constant | | Intermediate | |
| | | Maximum | | Value (b) | Maximum | Sum (sum of | |
| | Variable | Variable | | (Normal | Term Value | all previous | Over- |
| Term | Type | Value (a) | Variable Preprocessing | Mode) | (a * b) | rows) | flow |
| Eq2Offset | N/A | N/A | N/A | −194550 | −194550 | −194550 | NO |
| EqDScore * DCf | S32 | −503981 | The largest output for sub-equation D | 532 | −268117892 | −268312442 | NO |
| | | | may be −516077465 (see Table V). The | | | | |
| | | | input value may be scaled by dividing the | | | | |
| | | | value by 1024. Therefore the largest input | | | | |
| | | | value may be −503981 | | | | |
| Eq1ScorePrev * | S32 | −376000 | The largest output for sub-equation 1 may | 496 | −186496000 | −454808442 | NO |
| PrevEq1Cf | | | be −385024297 (see Table III). The input | | | | |
| | | | value may be scaled by dividing the value | | | | |
| | | | by 1024. Therefore the largest input value | | | | |
| | | | may be −376000 | | | | |
| Eq1ScoreCurrent * | S32 | −376000 | Same as previous row | 406 | −152656000 | −607464442 | NO |
| CurrEq1Cf | | | | | | | |

A qualification function may utilize the output of each of the equations discussed above (i.e., sub-equation 1, sub-equation D, and sub-equation 2) to facilitate qualification and/or rejection of a potential reciprocation. For example, the output of the qualification function may be filtered with an IIR filter, and the filtered output of the qualification function may be used to qualify or reject a reciprocation. An equation for an unfiltered qualification function output in accordance with present embodiments is set forth below:

QFUnfiltered=Eq1Score*SingleRecipWt*Eq2Cf+N2Score*MultipleRecipWt*Eq2Cf+NConsecRecip*ConsecCf+RecipMax*MaxCf+Artifact %*ArtCf+QFOffset,

where Eq2Cf, ConsecCf, MaxCf, ArtCf, and QFOffset may be selected using least squares analysis (e.g., using MATLAB®), and, as indicated above, Eq1Score may be defined as the output of sub-equation 1.

Other metrics in the unfiltered qualification function include SingleRecipWt, MultipleRecipWt, NConsecRecip, RecipMax, and Artifact %. With regard to SingleRecipWt and MultipleRecipWt, when there are two or more consecutive qualified reciprocations (e.g., qualified reciprocations that are less than 120 seconds apart) present, SingleRecipWt may equal 0 and MultipleRecipWt may equal 1. However, when only a single reciprocation is present, SingleRecipWt may equal 1 and MultipleRecipWt may equal 0.

NConseRecip, which may be defined as equal to max(NConsecRecip′,QFConsecMax), may include a count of the number of consecutive reciprocations (e.g., reciprocations that are less than or equal to 120 seconds apart) that have passed the hard limit checks. The value for NConsecRecip may be reset to 0 whenever a gap between any two partially qualified reciprocations exceeds 120 seconds. This may be based on the fact that ventilatory instability is a relatively long lasting event as compared to artifact. Therefore, as more reciprocations pass the hard limit checks, the qualification function may begin qualifying reciprocations that were previously considered marginal. However, to guard against a situation where something is causing a longer term artifact event (e.g., interference from nearby equipment), the value may be clipped to a maximum value to limit the metrics influence on the qualification function output.

RecipMax, which may be defined as equal to max(Fall Peak, Rise Peak), may facilitate making decisions about marginal reciprocations. Indeed, marginal reciprocations with higher maximum SpO₂ values may be more likely to get qualified than marginal reciprocations with lower SpO₂ values. It should be noted that this metric works in tandem with the NConsecRecip metric, and multiple marginal reciprocations with lower maximum SpO₂ values may eventually, over a long period of time, get qualified due to the NConsecRecip metric.

The metric Artifact % may be defined as an artifact percentage that is equal to 100*Total Artifact Count/Recip Duration, where Total Artifact Count is the number of times and artifact flag was set during the reciprocation. Present embodiments may include many metrics and equations that are used to set the artifact flag. Because of this it is a generally reliable indication of the amount of artifact present in the oximetry system as a whole. Marginal reciprocations with a high Artifact % are less likely to be qualified than marginal reciprocations with a low (or 0) artifact percentage.

A last component of the qualification function may include an infinite impulse response (IIR) filter that includes coefficients that may be tuned manually using a tool (e.g., a spreadsheet) that models algorithm performance. The filtered qualification function may be represented by the following equation, which includes different constants for different modes (e.g., Fast Response Mode and Normal Response Mode):

QFFiltered=SingleRecipWt*QFUnfiltered+((1−a)*QFUnfiltered+a*PrevQFFiltered)*MultipleRecipWt,

where QFUnfiltered may be defined as the current unfiltered qualification function output; PrevQFFiltered may be defined as the previous filtered qualification function output; and where the constant “a” may be set to 0.34 for Fast Response Mode and 0.5 for Normal Response Mode.

The filtered output of the qualification function may be compared to a threshold to determine if the current reciprocation is the result of RAF or artifact. The optimum threshold may theoretically be 0.5. However, an implemented threshold may be set slightly lower to bias the output of the qualification function towards qualifying more reciprocations, which may result in additional qualification of false positives. The threshold may be lowered because, in accordance with present embodiments, a cluster determination portion of the algorithm, such as may be performed by the CD feature 206, may require a certain number (e.g., 5) of fully qualified reciprocations before an index may be calculated, and a certain number (e.g., at least 2) of consecutive qualified reciprocations (with no intervening disqualified reciprocations) within the set of fully qualified reciprocations. Since multiple reciprocations may be required, the clustering detection method may be biased toward filtering out false positives. Accordingly, the reciprocation qualification function threshold may be lowered to balance the two processes.

The CD feature 206 may be capable of performing an algorithm that maintains an internal reciprocation counter that keeps track of a number of qualified reciprocations that are currently present. When the reciprocation counter is greater than or equal to a certain value, such as 5, the clustering state may be set to “active” and the algorithm may begin calculating and reporting the SPDi. When clustering is not active (e.g., reciprocation count<5) the algorithm may not calculate the SPDi. The SPDi may be defined as a scoring metric associated with the identification of a saturation trend pattern generated in accordance with present embodiment and may correlate to ventilatory instability in a population of sleep lab patients.

The CD feature 206 may utilize various rules to determine the reciprocation count. For example, when the clustering state is inactive, the following rules may be observed:
1.) If the distance between qualified reciprocation exceeds 120 seconds, then the reciprocation count=0;
2.) If the current reciprocation is qualified, and the time from the start of the current reciprocation to the end of the last qualified reciprocation is <=120 seconds, then the reciprocation count=reciprocation count+1;
3.) If the current reciprocation is not qualified, then the reciprocation count=max(reciprocation count−2, 0).
   Once clustering is active, it may remain active until the time between two qualified reciprocations exceeds 120 seconds. The following table (Table VIII) illustrates an example of how the reciprocation count rules may be applied to determine a clustering state.

TABLE VIII

| Current | Time | | |
|---|---|---|---|
| Reciprocation | Since Last Qualified | Reciprocation | Clustering |
| Qualified | Reciprocation (seconds) | Count | State |
| TRUE | N/A | 1 | INACTIVE |
| FALSE | 60 | 0 | INACTIVE |
| TRUE | N/A | 1 | INACTIVE |
| FALSE | 60 | 0 | INACTIVE |
| TRUE | N/A | 1 | INACTIVE |
| TRUE | 30 | 2 | INACTIVE |
| TRUE | 120 | 3 | INACTIVE |
| FALSE | 60 | 1 | INACTIVE |
| TRUE | 10 | 2 | INACTIVE |
| TRUE | 20 | 3 | INACTIVE |
| TRUE | 40 | 4 | INACTIVE |
| FALSE | 30 | 2 | INACTIVE |
| FALSE | 60 | 0 | INACTIVE |
| TRUE | N/A | 1 | INACTIVE |
| TRUE | 20 | 2 | INACTIVE |
| TRUE | 120 | 3 | INACTIVE |
| TRUE | 10 | 4 | INACTIVE |
| FALSE | 90 | 2 | INACTIVE |
| TRUE | 120 | 3 | INACTIVE |
| TRUE | 60 | 4 | INACTIVE |
| TRUE | 20 | 5 | ACTIVE |
| TRUE | 30 | 6 | ACTIVE |
| FALSE | 50 | 6 | ACTIVE |
| FALSE | 100 | 6 | ACTIVE |
| TRUE | 121 | 1 | INACTIVE |
| FALSE | 50 | 0 | INACTIVE |
| TRUE | N/A | 1 | INACTIVE |
| TRUE | 30 | 2 | INACTIVE |
| TRUE | 121 | 1 | INACTIVE |
| TRUE | 10 | 2 | INACTIVE |
| TRUE | 20 | 3 | INACTIVE |
| TRUE | 40 | 4 | INACTIVE |
| TRUE | 40 | 5 | ACTIVE |

When the clustering state is active, the SPDi calculation feature 208 may calculate an unfiltered SPDi for each new qualified reciprocation. The following formula may be used by the SPDi calculation feature 208:

Unfiltered SPDi=a*Magnitude+b*PeakDelta+c*NadirDelta;
wherein a=1.4, b=2.0, c=0.2;
wherein Magnitude=average magnitude of all reciprocations in the last 6 minutes;
wherein PeakDelta average of the three highest qualified reciprocation rise peaks in the last 6 minutes minus the average of the three lowest qualified reciprocation rise peaks in the last 6 minutes; and
wherein NadirDelta=average of the three highest qualified reciprocation nadirs in the last 6 minutes minus the average of the three lowest qualified reciprocation nadirs in the last 6 minutes.
Wherein SPDi<=31.

The above formula may be utilized to quantify the severity of a ventilatory instability pattern. The constants and metrics used may be based on input from clinical team members. It should be noted that the PeakDelta parameter may be assigned the largest weighting constant since the most severe patterns generally have peak reciprocation values that do not recover to the same baseline.

The unfiltered SPDi may be updated whenever clustering is active and a new qualified reciprocation is detected. Non-zero SPDi values may be latched for a period of time (e.g., 6 minutes). The unfiltered SPDi may then be low pass filtered to produce the final output SPDi value. The following IIR filter with a response time of approximately 40 seconds may be used:

SPDi=Unfiltered SPDi/a+Previous Filtered SPDi*(a−1)/a;

wherein a=40.

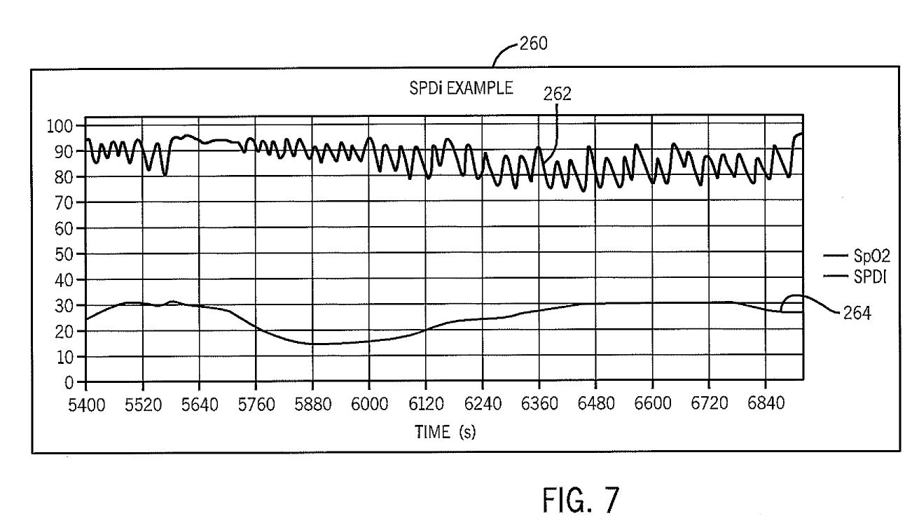

FIG. 7 is an exemplary graph 260 including an SpO₂ trend 262 that contains a ventilatory instability SpO₂ pattern and a trend of the resulting SPDi 264. In the illustrated example, it should be noted that the SPDi is sensitive to the decreasing peaks (incomplete recoveries) starting at approximately t=6000.

The UN feature 210 may be capable of determining if a user notification function should be employed to notify a user (e.g., via a graphical or audible indicator) of the presence of a detected patterns such as ventilatory instability. The determination of the UN feature 210 may be based on a user configurable tolerance setting and the current value of the SPDi. For example, the user may have four choices for the sensitivity or tolerance setting: Off, Low, Medium, and High. When the sensitivity or tolerance setting is set to Off, an alarm based on detection of a saturation pattern may never be reported to the user. The other three tolerance settings (i.e., Low, Medium, and High) may each map to an SPDi threshold value. For example, Low may map to an SPDi threshold of 6, Medium may map to an SPDi threshold of 15, and High may map to an SPDi threshold of 24. The thresholds may be based on input from users. When the SPDi is at or above the threshold for a given tolerance setting, the user may be notified that ventilatory instability is present. As discussed below, the indication to the user may include a graphical designation of the trend data corresponding to the detected pattern. For example, the trend data utilized to identify a ventilatory instability pattern may be highlighted, flashing, or otherwise indicated on a user interface of a monitor in accordance with present embodiments. Similarly, parameters such as the SPDi value and the tolerance settings may be graphically presented on a display.

It should be noted that, in order to detect certain data patterns, embodiments of the present disclosure may utilize systems and methods such as those disclosed in U.S. Pat. No. 6,760,608, U.S. Pat. No. 6,223,064, U.S. Pat. No. 5,398,682, U.S. Pat. No. 5,605,151, U.S. Pat. No. 6,748,252, U.S. application Ser. No. 11/455,408 filed Jun. 19, 2006, U.S. application Ser. No. 11/369,379 filed Mar. 7, 2006, and U.S. application Ser. No. 11/351,787 filed Feb. 10, 2006. Accordingly, U.S. Pat. No. 6,760,608, U.S. Pat. No. 6,223,064, U.S. Pat. No. 5,398,682, U.S. Pat. No. 5,605,151, U.S. Pat. No. 6,748,252, U.S. application Ser. No. 11/455,408 filed Jun. 19, 2006, U.S. application Ser. No. 11/369,379 filed Mar. 7, 2006, and U.S. application Ser. No. 11/351,787 filed Feb. 10, 2006 are each incorporated herein by reference.

Embodiments of the present disclosure may facilitate user observation and analysis of data, such as the detected patterns discussed above, by establishing a distinction between data of interest (e.g., data having certain notable characteristics, recent data) and other data (e.g., standard data, old data). For example, present embodiments may include graphical features that make a clear distinction between data detected within a designated time period (e.g., within 15 minutes) from a present time and data that is older (e.g., 15 minutes old or older). This may be beneficial in preventing a user (e.g., a clinician) from improperly diagnosing a current situation based on past data. Further, in another example, data of concern (e.g., data exhibiting a pattern of desaturation) may be distinguished from other data. The graphical features may include timestamps 104, graphic indicators 106, color changes in graphic features, flashing graphics, highlighting, blinking text, and so forth.

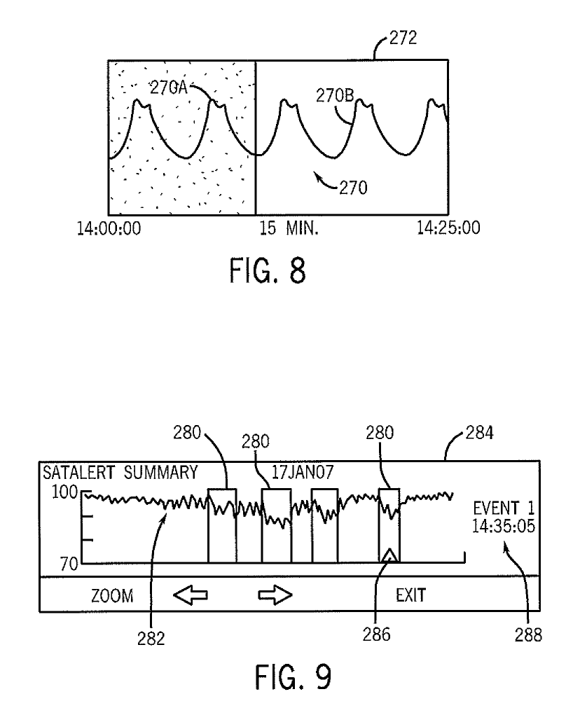

For example, as illustrated in FIG. 8, portions of a trend 270 in a trend display 272 that represent old data 270A (or data acquired over fifteen minutes before a present time) may be displayed as inverted, while current data 270B (or data acquired within fifteen minutes from the present time) may be displayed as normal. In another example, as illustrated in FIG. 9, detected patterns 280 in a trend 282 may be highlighted (or flashing) on a trend display 284 to distinguish the patterns 280 from other trend data. In some embodiments, if a particular pattern is of substantial interest it may flash, while other patterns may be simply highlighted. In yet other embodiments, the trend may be displayed in different colors or having varying line thicknesses depending on the nature (e.g., age and/or pattern) of the associated portions of trend data. Accordingly, when a user reviews trend data in accordance with present embodiments (e.g., snaps back or forward to an event), the user may readily discern the time period in which the event was recorded by observing the indicative graphical feature. It should be noted that in FIG. 9, an arrow 286 indicates that a particular pattern 280 has been selected and the time stamp 288 associated with the event is being displayed. In another embodiment, a vertical cursor line is used. In some embodiments, as will be discussed further below, a time scale may be presented along the trend 282 to facilitate identification of event occurrence times.

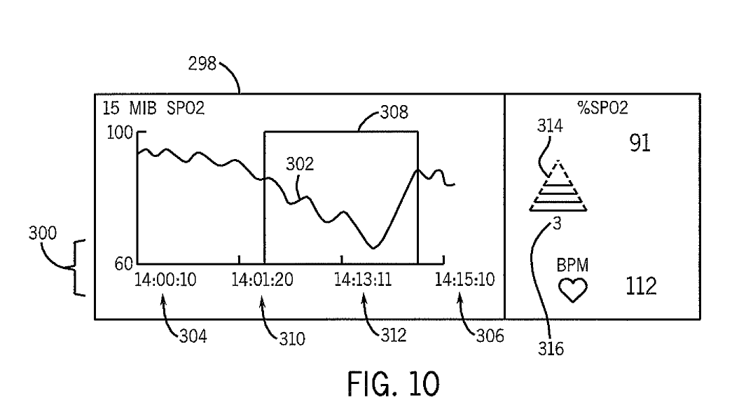

As suggested above, in addition to graphic identification of areas of interest in trend data, various other graphical and/or textual features may also facilitate user review of trend data. For example, as illustrated in the display screen 298 of FIG. 10, a time scale 300 may be displayed with respect to SpO₂ trend data 302 to avoid ambiguity as to when an event occurred. The time scale 300 may indicate the onset time 304 and the offset time 306 for the section of trend data being displayed. In some embodiments, onset and offset times may be displayed specifically for designated areas of interest within the trend data being displayed. For example, a highlighted portion 308 of the trend may have an onset time 310 and an offset time 312 at the beginning and end of the highlighted portion, respectively. It should be noted that in other embodiments, the time scale 300 may be utilized for different physiologic data trends (e.g., heart rate). Another feature that may facilitate user examination of monitor data is a status indicator 314 for pattern detection and/or severity, as illustrated in FIG. 10. In the illustrated embodiment, the status indicator 314 is represented as a triangle that may graphically fill from top to bottom as a monitored and/or calculated value increases. For example, in one embodiment, the status indicator 314 may gradually fill as the SPDi calculated by the SPDi calculation feature 208 increases. Further, the status indicator 314 may include a sensitivity level indicator 316 that displays a 1, 2, or 3, respectively, for sensitivity settings of High, Medium, and Low for the SPDi calculation feature 208.

As indicated above, various events in a trend of physiological data may be designated as being areas of interest by a device in accordance with present embodiments. For example, as discussed above, the monitor 10 may automatically detect and identify alarm events, saturation patterns in SpO₂ trend data, and so forth. Further, a user may utilize features of the monitor 10 to manually designate certain events. In view of the various events that may be designated in a data trend on the monitor 10, present embodiments may facilitate viewing these events without requiring a user to scroll through data that has not been identified as an area of interest. For example, a display control feature may be utilized to jump a display of a data trend to areas in the trend that have been automatically or manually designated as being of interest.

Activation of the display control feature during normal operation of the monitor 10 may cause the monitor 10 to jump or automatically scroll to a display of the most recent detected event. For example, in one embodiment, where no particular event type is designated, a user may press a button or the knob 50 to sequentially jump to all detected events in a set of historical data. Specifically, for example, with reference to FIG. 3, if no events are detected between the alarm 102 and when the display control feature is activated, activation of the display control feature may cause the monitor 10 to automatically display historical data of the trend 108 associated with the alarm 102. However, if events are detected between the time of the alarm and the time of activating the display control feature, the user may use the display control feature to cycle through the events to get to a display of data associated with the alarm 102. For example, a user may create the user designated event 112 by marking a certain portion of data at a point on the trend 108 after the alarm 102 occurred for later review. Such marking may be incorporated as an event by the monitor 10. Accordingly, activation of the display control feature from a current display may cause the monitor 10 to display the user designated event 112 (i.e., the marked data) before proceeding to display the data associated with the alarm 102, which would occur upon additional activation of the display control feature. Indeed, present embodiments may enable a user to cycle through all or a selected subset of events stored by the monitor 10.

A user may select different types of events for the display control feature to cycle through or jump to in accordance with present embodiments. In other words, the display control feature may be configured or programmed by the user such that activation of the display control feature causes the monitor's display to jump to specific types of events and to bypass others. This improves efficiency in viewing and analyzing data by allowing a user to skip over data that is irrelevant or not of interest. For example, a user may only be interested in alarms associated with recognized physiological patterns in the data (e.g., a pattern indicative of sleep apnea). Accordingly, the user may choose to view only labels that include alarms based on recognized physiological patterns and not labels based on equipment alarms (e.g., low battery alarms, sensor disconnected alarms), user markers, or other event types.

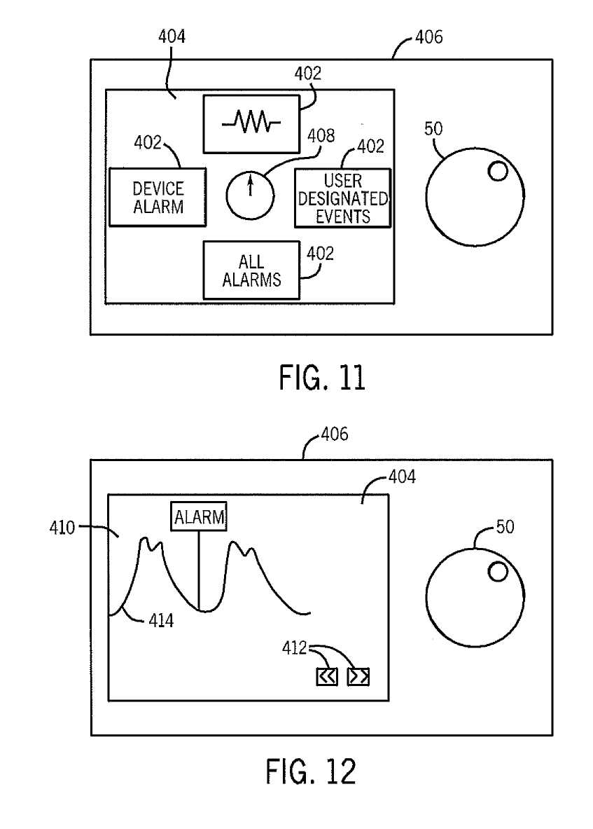

In some embodiments, a user may select particular types of events to snap or jump to when the display control feature is activated. For example, a user may turn the knob 50 to select between various soft menu features 402 that represent different types of events (e.g., events, data pattern types) in a display 404, as illustrated by the front view of a control panel 406 in FIG. 11. Turning the knob 50 may allow the user to navigate a menu or grouping of menu features 402 (e.g., buttons) and select the event type for the display control feature to seek out or jump to when it is activated. For example, a particular event type or set of event types may be selected by pressing the knob 50 when the button or menu item corresponding to the particular event type is highlighted or designated. In a specific example, a user may turn the knob 50 to guide a graphic arrow 408 such that it designates a desired one of the menu features 402, and the user may then depress the knob 50 to select the feature. If the user desires to deselect the feature, the process may be repeated to remove it as a selected feature. Once the event type or types are designated, the knob 50 may be utilized to navigate to a browsing menu 410, as illustrated in FIG. 12, which allows a user to select soft browsing buttons 412 by rotating the knob 50 to highlight the appropriate button and depressing the knob 50. The selection of the soft browsing buttons 412 may activate the display control feature and cause the display to jump to the most recent designated event type in the indicated direction within a trend 414 of historical data.

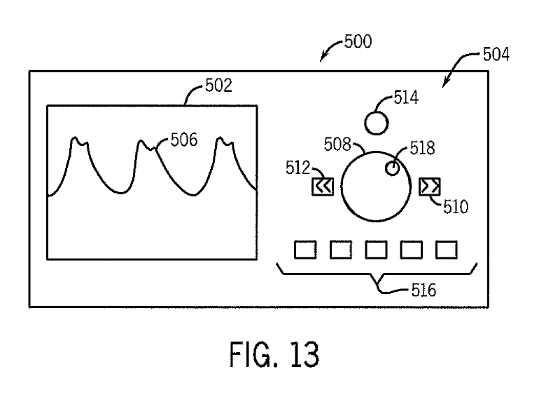

FIG. 13 is a front view of a control panel 500 in accordance with an exemplary embodiment of the present disclosure. Specifically, the control panel 500 includes a display screen 502 disposed adjacent a plurality of display control mechanisms 504. In the illustrated embodiment, the display screen 502 is displaying a trend 506 of data in an X-Y plot format. In other embodiments, different representations (e.g., bar graph, numerals, text) of the data may be employed. The control mechanisms 504 may include a dial 508, a find-forward button 510, a find-backward button 512, a select button 514, and/or a plurality of event designator buttons 516. The buttons may be actual buttons or soft buttons. While the illustrated embodiment shows the control mechanisms 504 on the faceplate of an actual monitor, in other embodiments, the control mechanisms 504 may be icons on a display screen and/or features disposed on a remote control that communicates with the actual monitor. In one embodiment, the entire control panel 500 may be a virtual control panel (e.g., a functional graphic) on a display presented on the display screen 502. It should be noted that if the display control feature is configured to only snap or jump to one type of event (e.g., detected desaturation patterns, or all detected events), the find-forward 510 and find-backward buttons 512 could be utilized without other features to simplify navigation of the historical data (e.g., trend 506).

The control mechanisms 504 may facilitate navigation through the history of the data (e.g., trend 506) represented on the display screen 502. For example, a user may rotate the dial 508 to slowly scroll through historical data recorded as the trend 506. The display of data may scroll in the direction that the dial 508 is rotated (i.e., counter-clockwise rotation of the dial scrolls the display back in time and clockwise rotation of the dial scrolls the display forward in time). The dial 508 may be substantially flush with the control panel 500, with a circular indentation 518 on the outer perimeter that facilitates rotation by allowing a user to insert a finger tip into the indentation 518 to control movement. In another example, the user may forgo scrolling through historical data by pressing the find-forward button 510 or the find-backward button 512, which may cause the display to jump to a certain event. In one embodiment, the view changes to include the most recent recognized event or selected event type in the direction indicated by the selected control mechanism 504 (e.g., find-backward button 512). For example, the monitor 10 may cause the screen 502 to display the last detected alarm when the find-backward button 512 is depressed or toggled from a real-time or standard operational display of the trending data 506. In another example, pressing the find-forward button 510 from a location in the historical data may cause the display to jump to the next recognized event or selected event type toward the present. If no events are identified between the location being observed and a real-time display, the display may simply jump to the real-time display.

The display control feature may be configured for selective viewing of labels using the event designator buttons 516 or similar input features. For example, a user may select one or more event designator buttons 516 that are associated with particular events of interest (e.g., alarms, alarm types, detected patterns, pattern types, user marks). In a specific example, a user may want the display control feature to operate such that when activated it cycles through sleep apnea patterns detected in a trend of physiological data. Accordingly, the user may select the event designator button 516 corresponding to detected sleep apnea patterns, thus causing the monitor 10 to jump directly to the display of these detected events when the display control feature is activated. In other examples, multiple event types may be selected for such observation. For example, multiple event designator buttons 516 may be activated such that the display control feature snaps to various alarm types and pattern types. Controlling the types of events that the monitor 10 automatically displays upon activation of the display control feature allows for efficient use of the monitor 10.

While the embodiments of the present disclosure may be susceptible to various modifications and alternative forms, specific embodiments have been shown by way of example in the drawings and will be described in detail herein. However, it should be understood that the present embodiments are not intended to be limited to the particular forms disclosed. Rather, present embodiments are to cover all modifications, equivalents and alternatives falling within the spirit and scope of present embodiments as defined by the following appended claims.

What is claimed is:

1. A monitoring system, comprising:
a monitor capable of receiving input relating to patient physiological parameters and storing historical data related to the parameters;
a display feature capable of displaying the historical data corresponding to the patient physiological parameters;
a pattern detection feature capable of analyzing the historical data to detect a physiologic pattern in a segment of the historical data and capable of designating a graphical representation of the segment with a graphical indication on the display feature when the physiologic pattern is present in the segment; and
a display control feature capable of automatically finding and displaying an event in the historical data on the screen when the display control feature is activated.

2. The system of claim 1, wherein the display control feature is capable of automatically finding and displaying user-specified types of events.

3. The system of claim 1, wherein the historical data comprises a trend of pulse oximetry data and the physiologic pattern comprises a desaturation pattern.

4. The system of claim 1, wherein the historical data comprises a trend of pulse oximetry data and the physiologic pattern comprises a pattern indicative of ventilatory instability and/or sleep apnea.

5. The system of claim 1, wherein the graphical indication comprises highlighting or flashing the graphical representation of the segment via the display feature.

6. The system of claim 1, wherein the graphical indication comprises a color coded indication of an importance level of the detected physiologic pattern.

7. The system of claim 1, comprising a saturation pattern detection index calculation feature capable of determining a scoring metric associated with the detected physiological pattern.

8. The system of claim 1, comprising a dynamic status indicator capable of indicating a status of pattern detection and/or an index level of a detected event.

9. The system of claim 8, wherein the dynamic status indicator comprises a graphic triangle capable of filling with a color from the bottom of the triangle to the top of the triangle as a saturation pattern detection index value increases based on the historical data.

10. A method, comprising:
receiving input relating to patient physiological parameters;
storing historical data related to the input;
detecting and graphically indicating a physiologic pattern in a displayed trend of the historical data; and
automatically scrolling to the detected physiologic pattern in the displayed trend when a display control feature is activated.

11. The method of claim 10, wherein graphically indicating the physiologic pattern comprises highlighting, flashing, and/or changing a color of a segment of the displayed trend that corresponds to the historical data identified as including the detected physiologic pattern.

12. The method of claim 10, comprising detecting the physiologic pattern with a limit-based qualification feature and a linear qualification feature.

13. The method of claim 10, wherein the historical data comprises pulse oximetry data and the pattern comprises a desaturation pattern.

14. The method of claim 10, comprising displaying a most recent data segment including a user-specified type of an identified pattern when a signal is received from the display control feature.

15. The method of claim 10, comprising displaying a time scale corresponding to the displayed trend.

16. The method of claim 10, comprising displaying a time scale corresponding to a beginning and an end of the physiologic pattern.

17. A method, comprising:
receiving physiological data from a sensor;
identifying a plurality of events in the physiological data;
determining whether a physiologic pattern is present in the physiological data based on whether the plurality of events meet defined criteria;
displaying the physiological data as a trend; and
graphically designating a segment of the trend with a graphic indicator that corresponds to an identified physiologic pattern.

18. The method of claim 17, wherein the graphic indicator comprises highlighting and/or flashing the segment of the trend.

19. The method of claim 17, comprising displaying a time scale corresponding to the trend.

20. The method of claim 18, comprising displaying a second time scale corresponding to the segment of the trend that corresponds to the identified physiologic pattern.
